(19) United States
(12) Patent Application Publication
(10) Pub. No.: US 2019/0046679 A1
(43) Pub. Date: Feb. 14, 2019

(54) Hygiene Compliance Method, Use and System

(71) Applicant: Skylark Indicators Limited, Leeds (GB)

(72) Inventors: Gregory A. Stoloff, London (GB); Robin M. Bannister, Essex (GB)

(73) Assignee: Skylark Indicators Limited, Leeds (GB)

(21) Appl. No.: 16/101,470

(22) Filed: Aug. 12, 2018

Related U.S. Application Data
(60) Provisional application No. 62/544,021, filed on Aug. 11, 2017.

(51) Int. Cl. A61L 2/28; C11D 11/00; C11D 17/04

(52) U.S. Cl. CPC A61L 2/28; G01N 21/29; C11D 17/04; C11D 11/0005

(57) ABSTRACT

The present specification discloses methods of monitoring hygiene compliance, hygiene monitoring systems and methods and uses of such systems.

The present application claims the benefit of priority and the filing date pursuant to 35 U.S.C. § 119(e) to U.S. Provisional Patent Application Ser. No. 62/544,021, filed on Aug. 11, 2017, the content of which is hereby incorporated by reference in its entirety.

Healthcare-associated infections (HAI), also referred to as nosocomial infections, is a major problem for patient safety, affecting 5% to 15% of hospitalized patients and 9% to 37% of those admitted to intensive care units. Unrelated to a patient's initial hospital admission diagnosis, HAI results in prolonged hospital stay, long-term disability, increased resistance of microorganisms to antimicrobials, massive additional financial burden, high costs for patients and their families, and excess deaths. It is estimated that each year in United States, over about 2 million hospitalized patients are affected by HAI, with about 99 000 deaths being attributed to these nosocomial infections. The annual economic impact of HAI in the U.S. ranges from $4.5 billion to $11 billion. Similarly, in Europe, approximately 5 million patients are affected by HAI each year, resulting in about 135,000 deaths and an economic burden of €13 billion to €24 billion. The HAI burden is greatly increased in high-risk patients such as those admitted to ICUs. Prevalence rates of infection acquired in ICUs vary from 9-37% in the U.S. and 9.7-31.8% in Europe, with crude mortality rates ranging from 12% to 80%.

More than 50% of all HAIs can be directly related to the transmission of harmful microorganisms by healthcare providers who have not properly washed their hands. To address this issue, the Centers for Disease Control and Prevention (CDC) as well as other regulatory agencies recommend hand washing before and after each patient encounter. Unfortunately, reports indicate that healthcare workers adhere to hand washing guidelines less than 70% of the time. Healthcare workers have reported several factors that can negatively impact their adherence with recommended hygiene practices including: 1) washing agents cause dryness, irritation and cracking to skin; 2) wash stations were inconveniently located; 3) lack of soap, paper towels or other cleaning supplies; 4) insufficient time to properly wash hands due to understaffing or overcrowding; 5) unfamiliar or disagree with recommend hygiene guidelines/protocols; 6) forgetfulness; 7) failure to realize how infrequent or inadequate worker complied with recommended hygiene practices; 8) low risk of acquiring infection from patients; 9) incomplete understanding of benefits of personal hygiene; and 10) patient needs taking priority.

Similar concerns exist in other industries, such as those relating to the processing and preparation of food. For example, the CDC estimates that each year food-borne microbial diseases cause approximately 76 million illnesses, 325,000 hospitalizations, and 5,000 deaths in the United States. To address this issue, the U.S. Food and Drug Administration's Food Code (the “Food Code”) provides guidelines for preparing food and preventing food-borne illness. Retail outlets such as restaurants and grocery stores and other institutions such as nursing homes are subject to the Food Code. In addition to requiring employees to wash their hands, the Food Code requires their employer to monitor the employees' hand washing. However, despite such extensive efforts to ensure hygiene compliance, the CDC estimated that improper hand washing was a contributing factor in over a third of the outbreaks.

Numerous strategies have been attempted to increase hygiene compliance as a preventative measure to HAIs and food-borne microbial diseases, but all have been largely unsuccessful. One major disadvantage of these strategies is expense and complex technologies employed. For example, many strategies require installation and use of sophisticated electronic monitoring technology to monitor and enforce hygiene compliance. Another disadvantage is that many of these strategies only allows for after-the-fact analysis; there is no real-time monitoring systems that enable instantaneous assessment of compliance. Furthermore, these strategies are essentially trust-based systems where personnel are entrusted to properly comply with recommended hygiene practices. Thus, unless personnel are actively supervised, there is usually no way to determine whether proper hygiene procedures have been followed. Furthermore, even if personnel do comply, there is no way to easily determine whether they have followed a prescribed government- and/or industry-approved regimen to ensure proper compliance.

Thus, there is an unmet need in the art for providing methods, uses and systems for monitoring and promoting proper hygiene practices. According the present specification discloses methods, uses and systems for monitoring hygiene compliance.

### SUMMARY

Aspects of the present specification disclose a method for monitoring hygiene compliance. In aspects, the disclosed method comprises the steps of applying a washing agent and a coloring agent to a surface area of an object; and detecting the presence or absence of color on the surface area applied with the washing agent over various different time scales. Certain aspects, of the disclosed method comprises the steps of applying a washing agent and a coloring agent to a skin surface of an individual; and detecting the presence or absence of color on the skin surface applied with the washing agent over various different specified time periods as applicable for the particular needs. In a preferred aspect, a washing agent and a coloring agent must be kept separate prior to application and applicator must have one depressing device that causes both agent and dye to be automatically dispensed at varying levels of each as required.

Other aspects of the present specification disclose a hygiene monitoring system. In aspects, the disclosed hygiene monitoring system comprises a washing agent and a coloring agent. A disclosed washing agent includes, without limitation, a personal washing agent, a residential washing agent, a commercial washing agent, or an industrial washing agent. A disclosed coloring agent includes, without limitation, a light-sensitive coloring agent, an ultraviolent light (UV)-sensitive coloring agent, an infrared (IR)-sensitive coloring agent, a pH-sensitive coloring agent, a redox-sensitive coloring agent, a chelating-sensitive coloring agent, an enzyme-sensitive coloring agent, a carbon dioxide-sensitive coloring agent, a thermo-sensitive coloring agent, or any combination thereof.

Other aspects of the present specification disclose use of a hygiene monitoring system disclosed herein for monitoring hygiene compliance.

Other aspects of the present specification disclose a hygiene monitoring system disclosed herein for use in monitoring hygiene compliance.

### DETAILED DESCRIPTION

A method for monitoring hygiene compliance comprising detecting the presence or absence of a coloring agent on a surface area of an object that was, or thought to have been, washed with a washing agent. The disclosed method comprises the steps of applying a washing agent and a coloring agent to a surface area of an object; and detecting the presence or absence of a coloring agent on the surface area washed with the washing agent. Thus, hygiene compliance is monitored based on detecting the presence or absence of a coloring agent on a surface area of the washed object.

A method for monitoring hygiene compliance comprising detecting the presence or absence of a coloring agent on a skin surface of an individual that was, or thought to have been, washed with a washing agent. The disclosed method comprises the steps of applying a washing agent and a coloring agent to a skin surface of an individual; and detecting the presence or absence of a coloring agent on the skin surface washed with the washing agent. Thus, hygiene compliance is monitored based on detecting the presence or absence of a coloring agent on a surface of the washed skin.

A hygiene monitoring system comprises a washing agent and a coloring agent. The disclosed system monitors hygiene compliance by detecting when a surface was, or thought to have been, washed with a washing agent.

The disclosed methods and systems enable the public to assess in real-time whether a person has complied with public health and safety standards, thereby making the consumer/patient the auditor of hygiene compliance. A resulting advantage is the ability to monitor hygiene compliance in a more cost effective and frequent manner than current systems and methods, which can involve expensive and bulky equipment.

The present specification discloses, in part, hygiene compliance. Hygiene relates to conditions or practices conducive to maintaining health and preventing disease, and includes, without limitation, personal hygiene, residential hygiene, commercial hygiene and industrial hygiene. Hygiene compliance relates to an action, state or fact of observing or satisfying a rule or standard pertaining to the conditions or practices conducive to maintaining health and preventing disease. Standards for hygiene compliance are promulgated by many public health and regulatory agencies including, without exception, World Health Organization (WHO), the CDC, as well as other regulatory agencies around the world.

The present specification discloses, in part, a washing agent. A washing agent refers to a substance used in establishing and/or maintaining hygiene, and includes, without limitation, a personal washing agent, a residential washing agent, a commercial washing agent and an industrial washing agent. A washing agent can establish and/or maintain hygiene by, e.g., reducing the number of microorganisms present on an object. A washing agent disclosed herein can be a broad-spectrum antimicrobial/antiseptic, fast-acting, and/or persistent. A washing agent may be in the form of a solid (i.e., bar of soap, surgical prep sponge), powder, liquid, cream, spray, gel, or the like. A washing agent disclosed herein includes, without limitation, a detergent, a soap, a cleaner, a bleach, a disinfectant, or a sterilizing agent.

A washing agent can be prepared in a wide variety of ways, including, e.g., a surfactant-based composition, an alcohol-based composition, an antimicrobial composition, an antiseptic composition, or any combination thereof. A surfactant-based composition refers to a preparation composed of both hydrophilic and lipophilic components and can be divided into four groups: anionic, cationic, amphoteric, and nonionic detergents. A surfactant-based composition includes, e.g., a detergent or a soap. An alcohol-based composition refers to a preparation comprising an alcohol, typically in an amount of about 50% to about 100%. Examples of commonly used alcohols include, without limitation, ethanol, isopropanol, n-propanol, or any combination thereof. An alcohol-based composition includes, e.g., an alcohol-based hand rub. An antimicrobial composition refers to a preparation comprising a washing agent and an antimicrobial agent. An antimicrobial composition includes, e.g., an antimicrobial hand wash, an antimicrobial hand rub, and a waterless antimicrobial composition. An antiseptic composition refers to a preparation comprising a washing agent and an antiseptic agent. An antiseptic composition includes, e.g., an antiseptic hand wash, an antiseptic hand rub, and a waterless antiseptic composition. An antiseptic agent includes, without limitation, an alcohol, chlorhexidine, chlorine, hexachlorophene, iodine, chloroxylenol (PCMX), a quaternary ammonium compound, and triclosan.

The present specification discloses, in part, a coloring agent. A coloring agent, also referred to as a colorant, is a dye, lake, pigment, or other substance that imparts a color to a surface area of an object and/or a skin surface of an individual at the same time as treatment with a washing agent. As such, a coloring agent disclosed herein is a surrogate indicator of use of a washing agent.

In one embodiment, a coloring agent disclosed herein produces a color that is permanent. A permanent color being produced by a coloring agent disclosed herein is one that cannot be removed without the use of a separate solvent. A coloring agent disclosed herein producing a permanent color serves as a visual indicator showing a location where a surface area of an object and/or a skin surface of an individual has been exposed to a washing agent. The coloring agent would then be removed from a surface area of an object and/or a skin surface of an individual using a cleaning agent designed to remove the coloring agent, such as, e.g., an oxidizing cleaner comprising an electrolyzed salt solution, such as, e.g., a hydrogen peroxide-based cleaner like SUPROX®. For example, floor cleaners typically require 10 minutes to 30 minutes of soak time by a washing agent in order to achieve the degree of disinfection needed to be hygiene compliant. Thus, a coloring agent would be removed from a surface area of an object and/or a skin surface of an individual after about 10 minutes to about 30 minutes has elapsed. As another example, an individual is required to wash his hands before and after each task, e.g., a healthcare provider examining patients throughout the day. Thus, a coloring agent would be removed only after the individual or another person, such as, e.g., a patient, consumer, patron, co-worker or supervisor, has acknowledged that his hands were properly washed, based on the presence of the coloring agent. Once proper acknowledgement has been obtained, the individual would remove the coloring agent, thereby ensuring that the next person, such as, e.g., a patient, consumer, patron, co-worker or supervisor, would know if the individual washed his hands just prior to this second visit.

In one embodiment, a coloring agent disclosed herein produces a color that is transient. A transient color being produced by a coloring agent disclosed herein is one that disappears after a certain time period without the need or use of a separate solvent. A coloring agent disclosed herein producing a transient color serves as a visual indicator showing a location where a surface area of an object and/or a skin surface of an individual has been exposed to a washing agent as well as the amount of time a surface area of an object and/or a skin surface of an individual has been exposed to a washing agent. The exact time period for which the coloring agent produces color will vary depending on the application or use. For example, floor cleaners typically require 10 minutes to 30 minutes of soak time by a washing agent in order to achieve the degree of disinfection needed to be hygiene compliant. Thus, a coloring agent selected would be one that produces color produced for about 10 minutes to about 30 minutes before disappearing. As another example, an individual is required to wash his hands before and after each task, e.g., a healthcare provider examining patients throughout the day. Thus, a coloring agent selected would be one that produces color that remains for about 2 minutes to about 5 minutes before disappearing. The presence of color on the hands would signify to the individual or another person, such as, e.g., a patient, consumer, patron, co-worker or supervisor, that his hands were properly washed. The color, however, would disappear during the examination, thereby ensuring that the next person, such as, e.g., a patient, consumer, patron, co-worker or supervisor, would know if the individual washed his hands just prior to this second visit.

In one embodiment, a coloring agent disclosed herein produces a permanent color or transient color after a certain period of time has elapsed. In aspects of this embodiment, a coloring agent produces a permanent color or transient color after, e.g., about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes. In other aspects of this embodiment, a coloring agent produces a permanent color or transient color after, e.g., at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes. In yet other aspects of this embodiment, a coloring agent produces a permanent color or transient color after, e.g., at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes. In still other aspects of this embodiment, a coloring agent produces color after, e.g., about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.

In one embodiment, a transient color produced by a coloring agent disclosed herein disappears after a certain period of time has elapsed. In aspects of this embodiment, a transient color produced by a coloring agent disclosed herein disappears after, e.g., about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes. In other aspects of this embodiment, a transient color produced by a coloring agent disappears after, e.g., at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes. In yet other aspects of this embodiment, a transient color produced by a coloring agent disclosed herein disappears after, e.g., at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes. In still other aspects of this embodiment, a transient color produced by a coloring agent disclosed herein disappears after, e.g., about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.

In other aspects of this embodiment, a coloring agent disclosed herein produces a transient color after, e.g., about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, and then the transient color disappears after, e.g., about 2 minutes to about 5 minutes, about 2 minutes to about 7 minutes, about 2 minutes to about 10 minutes, about 2 minutes to about 12 minutes, about 2 minutes to about 15 minutes, about 3 minutes to about 5 minutes, about 3 minutes to about 7 minutes, about 3 minutes to about 10 minutes, about 3 minutes to about 12 minutes, about 3 minutes to about 15 minutes, about 4 minutes to about 5 minutes, about 4 minutes to about 7 minutes, about 4 minutes to about 10 minutes, about 4 minutes to about 12 minutes, about 4 minutes to about 15 minutes, about 5 minutes to about 7 minutes, about 5 minutes to about 10 minutes, about 5 minutes to about 12 minutes, or about 5 minutes to about 15 minutes.

In other aspects of this embodiment, a coloring agent disclosed herein produces a transient color after, e.g., about 1 minute to about 5 minutes, about 1 minute to about 7 minutes, about 1 minute to about 10 minutes, about 1 minute to about 12 minutes, about 1 minute to about 15 minutes, about 2 minutes to about 5 minutes, about 2 minutes to about 7 minutes, about 2 minutes to about 10 minutes, about 2 minutes to about 12 minutes, about 2 minutes to about 15 minutes, about 3 minutes to about 5 minutes, about 3 minutes to about 7 minutes, about 3 minutes to about 10 minutes, about 3 minutes to about 12 minutes, about 3 minutes to about 15 minutes, about 4 minutes to about 5 minutes, about 4 minutes to about 7 minutes, about 4 minutes to about 10 minutes, about 4 minutes to about 12 minutes, about 4 minutes to about 15 minutes, about 5 minutes to about 7 minutes, about 5 minutes to about 10 minutes, about 5 minutes to about 12 minutes, or about 5 minutes to about 15 minutes, and then the transient color disappears after, e.g., about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.

A coloring agent disclosed herein may also have any one or combination of the following characteristics: (1) no intrinsic toxicity at concentrations required for detection; (2) generally recognized as a safe (GRAS) compound; (3) an U.S. Food and Drug Administration (FDA) approved chemical entity; (4) a good signal to noise (S:N) ratio that does not require a baseline reading; (5) has rapid onset of appearance or disappearance on surface of object after treatment with washing agent; (6) color detection is reproducible; (7) coloring agent is generated from a flexible chemistry formulation platform that will allow the selection of optimal coloring agents with regard to incorporation with washing agents and/or time of detection on surface of object. In addition, when used on the skin of a mammal, a coloring agent disclosed herein is safe for frequent use on the skin without the potential for allergic reactions, discoloration or drying of the skin.

The production of color using a coloring agent disclosed herein can be achieved using a wide array of physical or chemical triggers. For example, a coloring agent can be a light-sensitive coloring agent, an ultraviolent light (UV)-sensitive coloring agent, an infrared (IR)-sensitive coloring agent, a pH-sensitive coloring agent, a redox-sensitive coloring agent, a chelating-sensitive coloring agent, an enzyme-sensitive coloring agent, a carbon dioxide-sensitive coloring agent, or a thermo-sensitive coloring agent. A light sensitive coloring agent is one where coloration develops or disappears when the coloring agent is exposed to a specific threshold intensity of visible light for a certain amount of time. An UV-sensitive coloring agent is one where coloration develops or disappears when the coloring agent is exposed to a specific threshold intensity of UV light for a certain amount of time. An IR-sensitive coloring agent is one where coloration develops or disappears when the coloring agent is exposed to a specific threshold intensity IR light for a certain amount of time. A pH-sensitive coloring agent is one where coloration develops or disappears when the coloring agent is exposed to a specific threshold pH level for a certain amount of time. A redox-sensitive coloring agent is one where coloration develops or disappears when the coloring agent is exposed to a specific threshold of an oxidative species for a certain amount of time. A chelating-sensitive coloring agent is one where coloration develops or disappears when the coloring agent is exposed to a specific threshold of a chelator that sequesters a certain chemical species for a certain amount of time. An enzyme-sensitive coloring agent is one where coloration develops or disappears when the coloring agent undergoes an enzymatic reaction, either as the substrate or product. A carbon dioxide-sensitive coloring agent is one where coloration develops or disappears when the coloring agent is exposed to a specific threshold level of carbon dioxide. A thermo-sensitive coloring agent is one where coloration develops or disappears when the coloring agent is exposed to a specific threshold temperature for a certain amount of time.

The selection of a coloring agent disclosed herein can depend, in part, upon the intended purpose of the coloring agent, the time period the color should remain visible, the washing agent used, the surface to be washed, the length of time required for a proper washing, or any combination thereof. For example, floor cleaners typically require 10 minutes to 30 minutes of soak time by a washing agent in order to achieve the degree of disinfection needed to be hygiene compliant. Thus, a method disclosed herein would use a washing agent and a coloring agent where the color produced by the coloring agent would be present for about 10 minutes to about 30 minutes before disappearing. Elapse of the appropriate amount of time and/or the disappearance of color would be an indication that the floor surface was properly disinfected and the washing agent could not be removed from the floor surface.

As another example, proper hand washing requires that an individual wet thoroughly his hands and lathered with soap, rub the soapy water all over the hands and fingers for at least twenty (20) to thirty (30) seconds, including underneath the fingernails, and then rinse thoroughly to ensure proper washing has occurred. Thus, a method disclosed herein would use a washing agent and a coloring agent where the color is produced and remains for about 2 minutes to about 5 minutes before disappearing. The presence of color on the hands would signify to the individual or another person, such as, e.g., a patient, consumer, patron, co-worker or supervisor, that his hands were properly washed.

A coloring agent disclosed herein can be a food, cosmetic, and/or pharmaceutical grade coloring agent overseen by a governmental regulatory agency such as, e.g., the FDA. Such coloring agents include, without limitation, an acridine dye, an anthraquinone dye, an arylmethane dye, an azin dye, an azo dye, a diarylmethane dye, a diazonium dye, an eurhodin dye, a fluorene dye, an indigoid dye, an indamin dye, an indophenol dye, an Iodine-based dye, a lutein ester dye (mixture of carotenoid xanthophyll esters), a nitro dye, a nitroso dye, an oxazin dye, an oxazone dye, a phthalocyanine dye, a pyronin dye, a quinone-imine dye, a rhodamine dye, a safranin dye, a triarylmethane dye, a thiazine dye, a thiazole dye, and a xanthene dye. A coloring agent disclosed herein can also be an E124 synthetic blue dye, an E223 synthetic blue dye, an E162 synthetic red dye, a beet root red dye, a crystal violet, a gentian violet (also known as methyl violet 10B or hexamethyl pararosaniline chloride), a merbromin, a dihydroxyacetone (DHA), or any combination thereof.

A coloring agent disclosed herein can also be a thymolphthalein composition or a phenolphthalein composition. Such compositions typically include water and/or a buffer, sodium hydroxide and an alcohol, such as, e.g., ethanol. For example, in one embodiment, a thymolphthalein composition or a phenolphthalein composition comprises 1% thymolphthalein or phenolphthalein, 10% ethanol, 1% sodium hydroxide and water. Such formulations produce a dark blue solution (thymolphthalein composition) or a dark red solution (phenolphthalein composition). Upon exposure to air, carbon dioxide reacts with the water present in a thymolphthalein composition or a phenolphthalein composition to produce carbonic acid. The carbonic acid then reacts with the sodium hydroxide in a neutralization reaction to form sodium carbonate. This lowers the pH of the solution with the alcohol acting as an acid to turn the blue or red indicator colorless. The time needed for the color to disappear is controlled by the concentration of sodium hydroxide and/or a buffer present in the original composition. Such buffers include, without limitation, acetate buffers, citrate buffers, phosphate buffers, neutral buffered saline, phosphate buffered saline and borate buffers. It is understood that acids or bases can be used to adjust the pH of a composition as needed.

A coloring agent disclosed herein can be a free substance, microencapsulated, or coated or incorporated into particles. Microencapsulated particles used to a coloring agent can be made using standard microencapsulation techniques including, but not limited, coacervation (i.e., complex coacervation), double emulsion (w/o/w), polymerization (i.e., interfacial, in-situ, or matrix polymerization), co-extrusion capsule formulation (i.e., drip mode, centrifugal, or jet mode co-extrusion), and air-suspension coating. For instance, in the double emulsion technique, poly L-lactic acid (PLLA) is dissolved in methylene chloride, and emulsified with an aqueous (or other incompatible solvent) containing a coloring agent. This is adjusted to make a water in oil emulsion. This emulsion is poured into a second aqueous (or incompatible solvent) and air is bubbled through, which evaporates the organic solvent (methylene chloride), causing the PLLA to encapsulate a coloring agent in a hard shell. The skilled artisan would readily understand which polymers, solvents, temperature, stirring rates and surfactants should be added to optimize properties for detection in a washing agent. Capsules can be made to degrade or fracture under different conditions.

A coloring agent disclosed herein can be added directly to a washing agent disclosed herein, e.g., during manufacture of the washing agent, or subsequently incorporated into a washing agent already manufactured, e.g., by mixing a coloring agent immediately prior to or during application of the washing agent. However, one potential drawback with adding a coloring agent during the manufacturing process of a washing agent is that there is a likelihood that prolonged exposure of a coloring agent could change properties of a washing agent, particularly any antimicrobial properties, requiring reformulation and microbial testing.

In a preferred embodiment, mixing a color agent disclosed herein with a washing agent disclosed herein occurs just prior to or at the time of application. In this way, the washing agent and coloring agent are keep separate until needed, thereby maintaining the properties of the washing agent. In one embodiment, such mixing can be achieved simply by mixing a premeasured amount of a color agent to a a premeasured amount of washing agent, either before or after dilution with water or another dilutant.

In one embodiment, such mixing can be achieved using, e.g., a two-bottle system with a common dispensing mechanism. In this system, one bottle would contain the washing agent while the other bottle would contain the coloring agent. Upon operation, both the washing agent and coloring agent would be drawn up from their respective bottled and mixed together when the agents are expelled from the dispensing mechanism. Similarly, a dual chamber bottle system can be used. In this system, a single bottle comprises two chambers, with one chamber containing the washing agent while the other chamber containing the coloring agent. Upon operation, both the washing agent and coloring agent would be drawn up from their respective chambers and mixed together when the agents are expelled from the dispensing mechanism. Examples of bottle systems useful in mixing a color agent with a washing agent just prior to or at the time of application are described in, e.g., U.S. Pat. No. 9,586,714, U.S. Pat. No. 8,807,357, U.S. Pat. No. 8,746,475, U.S. Pat. No. 7,946,452, each of which is hereby incorporated by reference in its entirety. Commercially available bottle systems useful in mixing a color agent with a washing agent just prior to or at the time of application include the oval plastic dual chamber pump airless dispenser bottle LV-AJ017 (Levis International Trading Co., Ltd., Jiangsu, China).

An alternative mixing method is to presoak a washing agent contained in a solid substrate with a coloring agent just before use. For example, is a commercial setting, hand wipes are shipped in large containers containing thousands of wipes submerged in a washing agent. A coloring agent disclosed herein can simply be poured into these containers and allowed to diffuse and soak into the wipes before dispensing the wipes for use.

A color agent disclosed herein and a washing agent disclosed herein are mixed together for a desired amount of time to facilitate and/or ensure proper functionality. In aspects of this embodiment, a color agent and a washing agent are mixed together, e.g., about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes prior to application to a surface area of an object or a skin surface of an individual. In other aspects of this embodiment, a color agent and a washing agent are mixed together, e.g., at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes prior to application to a surface area of an object or a skin surface of an individual. In yet other aspects of this embodiment, a color agent and a washing agent are mixed together, e.g., at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes prior to application to a surface area of an object or a skin surface of an individual. In still other aspects of this embodiment, a color agent and a washing agent are mixed together, e.g., about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes prior to application to a surface area of an object or a skin surface of an individual.

The amount of coloring agent disclosed herein added to a washing agent disclosed herein is an amount sufficient to achieve the presence or absence of color desired to be detected. In aspects of this embodiment, the amount of coloring agent used is, e.g., about 0.01%, about 0.025%, about 0.05%, about 0.075%, about 0.1%, about 0.25%, about 0.5%, about 0.75%, about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, or about 35% by volume or weight of the total admixture of the coloring agent and washing agent. In other aspects of this embodiment, the amount of coloring agent used is, e.g., at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, or at least 35% by volume or weight of the total admixture of the coloring agent and washing agent. In yet other aspects of this embodiment, the amount of coloring agent used is, e.g., at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 20%, at most 25%, at most 30%, or at most 35% by volume or weight of the total admixture of the coloring agent and washing agent. In still other aspects of this embodiment, the amount of coloring agent used is, e.g., about 0.01% to about 0.05%, about 0.01% to about 0.1%, about 0.01% to about 0.5%, about 0.01% to about 1%, about 0.1% to about 0.5%, about 0.1% to about 1%, about 0.1% to about 10%, about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%, by volume or weight of the total admixture of the coloring agent and washing agent.

In other aspects of this embodiment, the ratio of washing agent to coloring agent used is, e.g., 10:1, 9:2, 9:1, 8:3, 8:1, 7:3, 7:2. 7:1, 6:1, 5:2, 5:1, 4:1, 3:2, 3:1, 2:1, or 1:1 washing agent:coloring agent.

Detecting the presence or absence of color can be after any length of time and is influenced, in part, by the intended purpose of the application, the coloring agent used, the washing agent used, the surface to be washed, the length of time required for a proper washing, or any combination thereof. In aspects of this embodiment, the presence or absence of color is detected after, e.g., about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes. In other aspects of this embodiment, the presence or absence of color is detected after, e.g., at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes. In yet other aspects of this embodiment, the presence or absence of color is detected after, e.g., at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes. In still other aspects of this embodiment, the presence or absence of color is detected after, e.g., about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.

The present specification discloses, in part, an object. An object is any material that can be seen and touched, including inanimate items as well as living animals. For example, an object can be a structure, an article of manufacture or surface from an animal. In aspects of this embodiment, an object can be, e.g., a floor, a wall, a ceiling, or any combination thereof. In other aspects of this embodiment, an object can be, e.g., a device, an equipment, an instrument a furniture, or any combination thereof. In other aspects of this embodiment, an object can be, e.g., a skin surface, a tooth surface, another body part from an animal, or any combination thereof. A skin surface includes, without limitation, a hand, an arm, a face, a scalp, a neck, a foot, a leg, a chest, a back or a buttock.

The present specification discloses, in part, washed or washing. Washed or washing refers to treating an object with a washing agent disclosed herein. For example, a floor, wall and/or ceiling of a room can be washed with a washing agent designed to clean, disinfect or sterilize the surfaces of the wall, floor and/or ceiling. Similarly, an article of manufacture, such as, e.g., a device, furniture or instrument, can be washed with a washing agent designed to clean, disinfect or sterilize the surfaces of the article of manufacture.

The present specification discloses, in part, hand washing. Hand washing, also known to as hand hygiene, refers to treating one or both hands of an individual with a hand washing agent designed to clean, disinfect or sterilize the skin of the hands. For example, the hands of an individual can be washed by applying soap and water to all surfaces of the hand, by applying only hand washing agent to all surfaces of the hand, by dipping hands into a hand washing agent or by using friction-generating activities to scrub the skin surface of the hands.

An object is cleaned by treating the object to remove dirt, marks or stains in order to produce an unsoiled object. Typically, an object is cleaned using plain (i.e., non-antimicrobial) detergent and water. A detergent (or soap) is a compound comprising one or more anionic, cationic, amphoteric, and/or nonionic surfactants.

An object is disinfected by treating an object to eliminate, render inert and/or prevent growth of many or all pathogenic microorganisms, but not necessarily all microbial forms, e.g., bacterial spores. Typically, an object is disinfected using a chemical-based composition, such as, e.g., an alcohol-based composition (like ethanol, isopropanol, and 2,4-dichlorobenzyl alcohol), an aldehyde-based composition (like glutaraldehyde and formaldehyde), an oxidizing-based composition (like a hypochlorate, a chloramine, an iodine, hydrogen peroxide, ozone, peracetic acid, performic acid, potassium permanganate or potassium peroxymonosulfate), a phenolic-based composition (like phenol, phenylphenol, chloroxylenol, hexachloroxylenol, thymol and amylmetacresol), a quaternary ammonium-based composition (like benzalkonium chloride), a silver-based or copper-based composition, ultraviolet irradiation, or by steaming.

An object is sterilized by treating an object to eliminate, remove, kill or deactivate all forms of life and other biological agents which are present, including, without limitation, fungi, bacteria, viruses, spore forms, prions, unicellular eukaryotic organisms. Sterilization can be achieved through various means, including, without exception, heat sterilization (such as, e.g., steam, dry heat, and flaming), chemical sterilization (such as, e.g., ethylene oxide, nitrogen dioxide, ozone, glutaraldehyde, formaldehyde, hydrogen peroxide and peracetic acid), irradiation (such as, e.g., non-ionizing radiation and ionizing radiation), high pressure sterilization (such as, e.g., pascalization), and filtration, or any combination thereof, such as, e.g., autoclaving which uses both heat and high pressure sterilization.

Aspects of the present specification may also be described as follows:
1. A method of monitoring hygiene compliance comprising: applying a washing agent and a coloring agent to a surface area of an object; and detecting the presence or absence of color on the surface area washed with the washing agent.
2. A method of monitoring hygiene compliance comprising: applying a washing agent and a coloring agent to a skin surface of an individual; and detecting the presence or absence of color on the skin surface washed with the washing agent.
3. The method according to Claim 1 or 2, wherein the washing agent is a personal washing agent, a residential washing agent, a commercial washing agent, or an industrial washing agent.
4. The method according to any one of Claims 1-3, wherein the washing agent is a detergent, a soap, a cleaner, a bleach, a disinfectant, or a sterilizing agent.
5. The method according to any one of Claims 1-4, wherein the washing agent is a surfactant-based composition, an alcohol-based composition, an antimicrobial composition, an antiseptic composition, or any combination thereof.
6. The method according to any one of Claims 1-5, wherein the coloring agent is a light-sensitive coloring agent, an ultraviolent light (UV)-sensitive coloring agent, an infrared (IR)-sensitive coloring agent, a pH-sensitive coloring agent, a redox-sensitive coloring agent, a chelating-sensitive coloring agent, an enzyme-sensitive coloring agent, a carbon dioxide-sensitive coloring agent, a thermo-sensitive coloring agent, or any combination thereof.
7. The method according to any one of Claims 1-6, wherein the coloring agent is an acridine dye, an anthraquinone dye, an arylmethane dye, an azin dye, an azo dye, a diarylmethane dye, a diazonium dye, an eurhodin dye, a fluorene dye, an indigoid dye, an indamin dye, an indophenol dye, an Iodine-based dye, a lutein ester dye (mixture of carotenoid xanthophyll esters), a nitro dye, a nitroso dye, an oxazin dye, an oxazone dye, a phthalocyanine dye, a pyronin dye, a quinone-imine dye, a rhodamine dye, a safranin dye, a triarylmethane dye, a thiazine dye, a thiazole dye, a xanthene dye, an E124 synthetic blue dye, an E223 synthetic blue dye, an E162 synthetic red dye, a beet root red dye, a crystal violet, a gentian violet, a merbromin, a dihydroxyacetone, or any combination thereof.
8. The method according to any one of Claims 1-7, wherein the coloring agent is a thymolphthalein composition or a phenolphthalein composition.
9. The method according to any one of Claims 1-8, wherein the coloring agent is a free substance, microencapsulated, coated or incorporated into a particle, or any combination thereof.
10. The method according to any one of Claim 1 or 3-9, wherein the washing agent and coloring agent are mixed together immediately prior to or during application the surface area.
11. The method according to any one of Claims 2-9, wherein the washing agent and coloring agent are mixed together immediately prior to or during application to the skin surface.
12. The method according to Claim 10 or 11, wherein the washing agent and coloring agent are mixed together about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes prior to application to a surface area of an object or a skin surface of an individual; or at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes prior to application to a surface area of an object or a skin surface of an individual; or at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes prior to application to a surface area of an object or a skin surface of an individual; or about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes prior to application to a surface area of an object or a skin surface of an individual.
13. The method according to any one of Claims 1-12, wherein the amount of coloring agent applied is about 0.01%, about 0.025%, about 0.05%, about 0.075%, about 0.1%, about 0.25%, about 0.5%, about 0.75%, about 1%, about 2%, about 3%, about 4%, about 5%, about 6%, about 7%, about 8%, about 9%, about 10%, about 11%, about 12%, about 13%, about 14%, about 15%, about 20%, about 25%, or about 30% by volume or weight of the total admixture of the coloring agent and washing agent; or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, or at least 30% by volume or weight of the total admixture of the coloring agent and washing agent; or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 20%, at most 25%, or at most 30% by volume or weight of the total admixture of the coloring agent and washing agent; or about 0.01% to about 0.05%, about 0.01% to about 0.1%, about 0.01% to about 0.5%, about 0.01% to about 1%, about 0.1% to about 0.5%, about 0.1% to about 1%, about 0.1% to about 10%, about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%, by volume or weight of the total admixture of the coloring agent and washing agent.
14. The method according to any one of Claims 1-13, wherein the ratio of washing agent to coloring agent used is 10:1, 9:2, 9:1, 8:3, 8:1, 7:3, 7:2. 7:1, 6:1, 5:2, 5:1, 4:1, 3:2, 3:1, 2:1, or 1:1 washing agent:coloring agent.
15. The method according to any one of Claim 1 or 3-10, or 12-14, wherein the object is a structure, an article of manufacture or surface from an animal.
16. The method according to Claim 15, wherein the structure is a floor, a wall, a ceiling, or any combination thereof.
17. The method according to Claim 15, wherein the article of manufacture is a device, an equipment, an instrument a furniture, or any combination thereof.
18. The method according to Claim 15, wherein the surface from the animal is a skin surface, a tooth surface, another body part from a mammal, or any combination thereof.
19. The method according to any one of Claim 2-9, 11-14 or 18, wherein the skin surface comprises, a hand, an arm, a face, a scalp, a neck, a foot, a leg, a chest, a back, a buttock, or any combination thereof.
20. The method according to any one of Claims 1-19, wherein the coloring agent produces color after about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes; or after at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes; or after at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes; or after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
21. The method according to any one of Claims 1-20, wherein the color produced by a coloring agent disappears after about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes; or after at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes; or after at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes; or after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
22. The method according to any one of Claims 1-19, wherein the coloring agent produces color after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, and then the color disappears after about 2 minutes to about 5 minutes, about 2 minutes to about 7 minutes, about 2 minutes to about 10 minutes, about 2 minutes to about 12 minutes, about 2 minutes to about 15 minutes, about 3 minutes to about 5 minutes, about 3 minutes to about 7 minutes, about 3 minutes to about 10 minutes, about 3 minutes to about 12 minutes, about 3 minutes to about 15 minutes, about 4 minutes to about 5 minutes, about 4 minutes to about 7 minutes, about 4 minutes to about 10 minutes, about 4 minutes to about 12 minutes, about 4 minutes to about 15 minutes, about 5 minutes to about 7 minutes, about 5 minutes to about 10 minutes, about 5 minutes to about 12 minutes, or about 5 minutes to about 15 minutes.
23. The method according to any one of Claims 1-19, wherein the coloring agent produces color after about 1 minute to about 5 minutes, about 1 minute to about 7 minutes, about 1 minute to about 10 minutes, about 1 minute to about 12 minutes, about 1 minute to about 15 minutes, about 2 minutes to about 5 minutes, about 2 minutes to about 7 minutes, about 2 minutes to about 10 minutes, about 2 minutes to about 12 minutes, about 2 minutes to about 15 minutes, about 3 minutes to about 5 minutes, about 3 minutes to about 7 minutes, about 3 minutes to about 10 minutes, about 3 minutes to about 12 minutes, about 3 minutes to about 15 minutes, about 4 minutes to about 5 minutes, about 4 minutes to about 7 minutes, about 4 minutes to about 10 minutes, about 4 minutes to about 12 minutes, about 4 minutes to about 15 minutes, about 5 minutes to about 7 minutes, about 5 minutes to about 10 minutes, about 5 minutes to about 12 minutes, or about 5 minutes to about 15 minutes, and then the color disappears after about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
24. The method according to any one of Claims 1-23, wherein the presence or absence of color is detected after about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes; or after at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes; or after at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes; or after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
25. The method according to any one of Claims 1-24, wherein the hygiene compliance being monitored comprises personal hygiene, residential hygiene, commercial hygiene, industrial hygiene, or any combination thereof.
26. A hygiene monitoring system comprises a washing agent and a coloring agent.
27. The system according to Claim 26, wherein the washing agent is a personal washing agent, a residential washing agent, a commercial washing agent, or an industrial washing agent.
28. The system according to Claim 26 or 27, wherein the washing agent is a detergent, a soap, a cleaner, a bleach, a disinfectant, or a sterilizing agent.
29. The system according to any one of Claims 26-28, wherein the washing agent is a surfactant-based composition, an alcohol-based composition, an antimicrobial composition, an antiseptic composition, or any combination thereof.
30. The system according to any one of Claims 26-29, wherein the coloring agent is a light-sensitive coloring agent, an ultraviolent light (UV)-sensitive coloring agent, an infrared (IR)-sensitive coloring agent, a pH-sensitive coloring agent, a redox-sensitive coloring agent, a chelating-sensitive coloring agent, an enzyme-sensitive coloring agent, a carbon dioxide-sensitive coloring agent, a thermo-sensitive coloring agent, or any combination thereof.
31. The system according to any one of Claims 26-30, wherein the coloring agent is an acridine dye, an anthraquinone dye, an arylmethane dye, an azin dye, an azo dye, a diarylmethane dye, a diazonium dye, an eurhodin dye, a fluorene dye, an indigoid dye, an indamin dye, an indophenol dye, an Iodine-based dye, a lutein ester dye (mixture of carotenoid xanthophyll esters), a nitro dye, a nitroso dye, an oxazin dye, an oxazone dye, a phthalocyanine dye, a pyronin dye, a quinone-imine dye, a rhodamine dye, a safranin dye, a triarylmethane dye, a thiazine dye, a thiazole dye, a xanthene dye, an E124 synthetic blue dye, an E223 synthetic blue dye, an E162 synthetic red dye, a beet root red dye, a crystal violet, a gentian violet, a merbromin, a dihydroxyacetone, or any combination thereof.
32. The system according to any one of Claims 26-31, wherein the coloring agent is a thymolphthalein composition or a phenolphthalein composition.
33. The system according to any one of Claims 26-32, wherein the coloring agent is a free substance, microencapsulated, coated or incorporated into a particle, or any combination thereof.
34. Use of a hygiene monitoring system as defined in any one of Claims 26-33 for monitoring hygiene compliance.
35. A hygiene monitoring system as defined in any one of Claims 26-33 for use to monitor hygiene compliance.
36. The use according to Claim 34 or 35, wherein the hygiene compliance being monitored comprises personal hygiene, residential hygiene, commercial hygiene, industrial hygiene, or any combination thereof.
37. A method of monitoring hygiene compliance comprising: applying a hygiene monitoring system as defined in any one of Claims 26-33 to a surface area of an object; and detecting the presence or absence of color on the surface area washed with the washing agent.
38. The method according to Claim 37 wherein the washing agent and coloring agent are mixed together immediately prior to or during application the surface area.
39. The method according to Claim 37 or 38, wherein the object is a structure, an article of manufacture or surface from an animal.
40. The method according to Claim 39, wherein the structure is a floor, a wall, a ceiling, or any combination thereof.
41. The method according to Claim 39, wherein the article of manufacture is a device, an equipment, an instrument a furniture, or any combination thereof.
42. The method according to Claim 39, wherein the surface from a mammal is a skin surface, a tooth surface, another body part from an animal, or any combination thereof.
43. A method for monitoring hygiene compliance comprising: applying a hygiene monitoring system as defined in any one of Claims 26-33 to a skin surface of an individual; and detecting the presence or absence of color on the skin surface washed with the washing agent.
44. The method according to Claim 43, wherein the washing agent and coloring agent are mixed together immediately prior to or during application to the skin surface.
45. The method according to Claim 43 or 44, wherein the skin surface comprises, a hand, an arm, a face, a scalp, a neck, a foot, a leg, a chest, a back, a buttock, or any combination thereof.
46. The method according to any one of Claims 37-45, wherein the washing agent and coloring agent are mixed together about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes prior to application to a surface area of an object or a skin surface of an individual; or at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes prior to application to a surface area of an object or a skin surface of an individual; or at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes prior to application to a surface area of an object or a skin surface of an individual; or about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes prior to application to a surface area of an object or a skin surface of an individual.
47. The method according to any one of Claims 37-46, wherein the amount of coloring agent applied is about 0.01%, about 0.025%, about 0.05%, about 0.075%, about 0.1%, about 0.25%, about 0.5%, about 0.75%, about 1%, about 2%, about 3%, about 4%, about 5%, about 6%, about 7%, about 8%, about 9%, about 10%, about 11%, about 12%, about 13%, about 14%, about 15%, about 20%, about 25%, or about 30% by volume or weight of the total admixture of the coloring agent and washing agent; or at least 0.01%, at least 0.025%, at least 0.05%, at least 0.075%, at least 0.1%, at least 0.25%, at least 0.5%, at least 0.75%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, or at least 30% by volume or weight of the total admixture of the coloring agent and washing agent; or at most 0.01%, at most 0.025%, at most 0.05%, at most 0.075%, at most 0.1%, at most 0.25%, at most 0.5%, at most 0.75%, at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 20%, at most 25%, or at most 30% by volume or weight of the total admixture of the coloring agent and washing agent; or about 0.01% to about 0.05%, about 0.01% to about 0.1%, about 0.01% to about 0.5%, about 0.01% to about 1%, about 0.1% to about 0.5%, about 0.1% to about 1%, about 0.1% to about 10%, about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30%, by volume or weight of the total admixture of the coloring agent and washing agent.
48. The method according to any one of Claims 37-47, wherein the ratio of washing agent to coloring agent used is 10:1, 9:2, 9:1, 8:3, 8:1, 7:3, 7:2. 7:1, 6:1, 5:2, 5:1, 4:1, 3:2, 3:1, 2:1, or 1:1 washing agent:coloring agent.
49. The method according to any one of Claims 37-48, wherein the coloring agent produces color after about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes; or after at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes; or after at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes; or after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
50. The method according to any one of Claims 37-49, wherein the color produced by a coloring agent disappears after about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes; or after at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes; or after at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes; or after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
51. The method according to any one of Claims 37-48, wherein the coloring agent produces color after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, and then the color disappears after about 2 minutes to about 5 minutes, about 2 minutes to about 7 minutes, about 2 minutes to about 10 minutes, about 2 minutes to about 12 minutes, about 2 minutes to about 15 minutes, about 3 minutes to about 5 minutes, about 3 minutes to about 7 minutes, about 3 minutes to about 10 minutes, about 3 minutes to about 12 minutes, about 3 minutes to about 15 minutes, about 4 minutes to about 5 minutes, about 4 minutes to about 7 minutes, about 4 minutes to about 10 minutes, about 4 minutes to about 12 minutes, about 4 minutes to about 15 minutes, about 5 minutes to about 7 minutes, about 5 minutes to about 10 minutes, about 5 minutes to about 12 minutes, or about 5 minutes to about 15 minutes.
52. The method according to any one of Claims 37-48, wherein the coloring agent produces color after about 1 minute to about 5 minutes, about 1 minute to about 7 minutes, about 1 minute to about 10 minutes, about 1 minute to about 12 minutes, about 1 minute to about 15 minutes, about 2 minutes to about 5 minutes, about 2 minutes to about 7 minutes, about 2 minutes to about 10 minutes, about 2 minutes to about 12 minutes, about 2 minutes to about 15 minutes, about 3 minutes to about 5 minutes, about 3 minutes to about 7 minutes, about 3 minutes to about 10 minutes, about 3 minutes to about 12 minutes, about 3 minutes to about 15 minutes, about 4 minutes to about 5 minutes, about 4 minutes to about 7 minutes, about 4 minutes to about 10 minutes, about 4 minutes to about 12 minutes, about 4 minutes to about 15 minutes, about 5 minutes to about 7 minutes, about 5 minutes to about 10 minutes, about 5 minutes to about 12 minutes, or about 5 minutes to about 15 minutes, and then the color disappears after about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
53. The method according to any one of Claims 37-52, wherein the presence or absence of color is detected after about 5 seconds, about 10 seconds, about 15 seconds, about 30 seconds, about 45 seconds, about 60 seconds, about 2 minutes, about 3 minutes, about 4 minutes, about 5 minutes, about 10 minutes, about 15 minutes, about 20 minutes, about 25 minutes or about 30 minutes; or after at least 5 seconds, at least 10 seconds, at least 15 seconds, at least 30 seconds, at least 45 seconds, at least 60 seconds, at least 2 minutes, at least 3 minutes, at least 4 minutes, at least 5 minutes, at least 10 minutes, at least 15 minutes, at least 20 minutes, at least 25 minutes or at least 30 minutes; or after at most 5 seconds, at most 10 seconds, at most 15 seconds, at most 30 seconds, at most 45 seconds, at most 60 seconds, at most 2 minutes, at most 3 minutes, at most 4 minutes, at most 5 minutes, at most 10 minutes, at most 15 minutes, at most 20 minutes, at most 25 minutes or at most 30 minutes; or after about 5 seconds to about 10 seconds, about 5 seconds to about 15 seconds, about 5 seconds to about 30 seconds, about 5 seconds to about 45 seconds, about 5 seconds to about 60 seconds, about 10 seconds to about 15 seconds, about 10 seconds to about 30 seconds, about 10 seconds to about 45 seconds, about 10 seconds to about 60 seconds, about 30 seconds to about 45 seconds, about 30 seconds to about 60 seconds, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes.
54. The method according to any one of Claims 37-53, wherein the hygiene compliance being monitored comprises personal hygiene, residential hygiene, commercial hygiene, industrial hygiene, or any combination thereof.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the methods, systems and uses disclosed herein.

### Example 1

### Method of Cleaning a Surface

A janitor is assigned to clean the floors of a maternity ward of a healthcare facility. The floor cleaning product used by the healthcare facility is a disinfectant that must soak on the floor for 15 minutes before removal in order to achieve maximum disinfection of the floor surface. Just before use, the janitor dispenses the floor cleaning product in a bucket using a system that automatically adds the appropriate amount of coloring agent, e.g., a phenolphthalein composition. The janitor then mops the floor with the admixture. Within 30 seconds upon application of the cleaning product admixture to the floor, a red color develops. This red color remains visible for 15-20 minutes. Upon disappearance of the red color, the janitor mops of the floor cleaning product admixture to complete the cleaning process.

### Example 2

### Method of Cleaning Hands

A healthcare provider working in an office suite is tasked to take the vital signs of each patient as a preliminary examination before a healthcare physician examines the patient. Before entering an examination room to take the signs of the next patient, the healthcare provider washes her hands with a disinfectant that admixes a coloring agent disclosed herein, e.g. a thymolphthalein composition, upon dispensing onto her hands. After rubbing the disinfectant all over her hands and fingers for at least 20-30 seconds, including underneath the fingernails, a blue color appears on her hands. The blue color remains after rinsing her hands thoroughly with water but will eventually fade away after 2-3 minutes. Upon entering the examination room the patient sees that the healthcare provider's hands are blue and can make a note that her hands were properly washed (the patient knows this as there are signs posted in the examination room stating that all staff examining the patient must have green stained hands upon entering the room).

### Example 3

### Method of Cleaning a Surface

A butcher working in a supermarket is tasked to clean the table surfaces used during the day to prepare meats for the customers. The surface cleaning product used by the butcher is a detergent that must soak on the table surfaces for 10 minutes before removal in order to achieve maximum cleansing effect and disinfection of the surface. Just before use, the butcher dispenses the surface cleaning product in a bucket using a system that automatically adds the appropriate amount of coloring agent, e.g., an E162 red dye. In one application, about 150 mg of an E162 red dye is added to 25 mL of a non-oxidizing surface cleaning product, such as, e.g., Proteus Degreaser. The butcher then cleans the table surfaces with the admixture, applying enough to allow the surfaces to soak with the admixture. Within 1 minute upon application of the cleaning product admixture to the surfaces, a pink color develops. This pink color remains visible for 10-15 minutes. Upon disappearance of the pink color, the butcher wipes down of the surface cleaning product admixture to complete the cleaning process.

### Example 4

### Method of Cleaning a Surface

A housewife decides to clean the surfaces of a bathroom. The surface cleaning product used by the housewife is a detergent that must soak on the table surfaces for 10 minutes before removal in order to achieve maximum cleansing effect and disinfection of the surface. Just before use, the housewife dispenses the surface cleaning product in a bucket using a system that automatically adds the appropriate amount of coloring agent, e.g., an E124 blue dye. In one application, about 15 mg of an E124 blue dye is added to 25 mL of a non-oxidizing surface cleaning product, such as, e.g., Proteus Essential Cleaner. The housewife then cleans the surfaces of the bathroom with the admixture. Within 15 seconds upon application of the cleaning product admixture to the surfaces, a blue color develops. After 10-15 minutes the housewife then uses using a separate solution made up of an oxidizing cleaner to remove the blue color off the surfaces to complete the cleaning process. In one application, oxidizing cleaner used is an electrolyzed salt solution, such as, e.g., a hydrogen peroxide-based cleaner like SUPROX®.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A method of monitoring hygiene compliance comprising:
applying a washing agent and a coloring agent to a surface area of an object; and
detecting the presence or absence of color on the surface area washed with the washing agent.

2. The method according to claim 1, wherein the washing agent is a personal washing agent, a residential washing agent, a commercial washing agent, or an industrial washing agent.

3. The method according to claim 1, wherein the washing agent is a detergent, a soap, a cleaner, a bleach, a disinfectant, or a sterilizing agent.

4. The method according to claim 1, wherein the washing agent is a surfactant-based composition, an alcohol-based composition, an antimicrobial composition, an antiseptic composition, or any combination thereof.

5. The method according to claim 1, wherein the coloring agent is a light-sensitive coloring agent, an ultraviolent light (UV)-sensitive coloring agent, an infrared (IR)-sensitive coloring agent, a pH-sensitive coloring agent, a redox-sensitive coloring agent, a chelating-sensitive coloring agent, an enzyme-sensitive coloring agent, a carbon dioxide-sensitive coloring agent, a thermo-sensitive coloring agent, or any combination thereof.

6. The method according to claim 1, wherein the coloring agent is an acridine dye, an anthraquinone dye, an arylmethane dye, an azin dye, an azo dye, a diarylmethane dye, a diazonium dye, an eurhodin dye, a fluorene dye, an indigoid dye, an indamin dye, an indophenol dye, an Iodine-based dye, a lutein ester dye (mixture of carotenoid xanthophyll esters), a nitro dye, a nitroso dye, an oxazin dye, an oxazone dye, a phthalocyanine dye, a pyronin dye, a quinone-imine dye, a rhodamine dye, a safranin dye, a triarylmethane dye, a thiazine dye, a thiazole dye, a xanthene dye, an E124 synthetic blue dye, an E223 synthetic blue dye, an E162 synthetic red dye, a beet root red dye, a crystal violet, a gentian violet, a merbromin, a dihydroxyacetone, or any combination thereof.

7. The method according to claim 1, wherein the coloring agent is a free substance, microencapsulated, coated or incorporated into a particle, or any combination thereof.

8. The method according to claim 1, wherein the washing agent and coloring agent are mixed together immediately prior to or during application the surface area.

9. The method according to claim 1, wherein the object is a structure, an article of manufacture or surface from an animal.

10. The method according to claim 1, wherein the hygiene compliance being monitored comprises personal hygiene, residential hygiene, commercial hygiene, industrial hygiene, or any combination thereof.

11. A hygiene monitoring system comprises a washing agent and a coloring agent.

12. The system according to claim 11, wherein the washing agent is a personal washing agent, a residential washing agent, a commercial washing agent, or an industrial washing agent.

13. The system according to claim 11, wherein the washing agent is a detergent, a soap, a cleaner, a bleach, a disinfectant, or a sterilizing agent.

14. The system according to claim 11, wherein the washing agent is a surfactant-based composition, an alcohol-based composition, an antimicrobial composition, an antiseptic composition, or any combination thereof.

15. The system according to claim 11, wherein the coloring agent is a light-sensitive coloring agent, an ultraviolent light (UV)-sensitive coloring agent, an infrared (IR)-sensitive coloring agent, a pH-sensitive coloring agent, a redox-sensitive coloring agent, a chelating-sensitive coloring agent, an enzyme-sensitive coloring agent, a carbon dioxide-sensitive coloring agent, a thermo-sensitive coloring agent, or any combination thereof.

16. The system according to claim 11, wherein the coloring agent is an acridine dye, an anthraquinone dye, an arylmethane dye, an azin dye, an azo dye, a diarylmethane dye, a diazonium dye, an eurhodin dye, a fluorene dye, an indigoid dye, an indamin dye, an indophenol dye, an Iodine-based dye, a lutein ester dye (mixture of carotenoid xanthophyll esters), a nitro dye, a nitroso dye, an oxazin dye, an oxazone dye, a phthalocyanine dye, a pyronin dye, a quinone-imine dye, a rhodamine dye, a safranin dye, a triarylmethane dye, a thiazine dye, a thiazole dye, a xanthene dye, an E124 synthetic blue dye, an E223 synthetic blue dye, an E162 synthetic red dye, a beet root red dye, a crystal violet, a gentian violet, a merbromin, a dihydroxyacetone, or any combination thereof.

17. The system according to claim 11, wherein the coloring agent is a free substance, microencapsulated, coated or incorporated into a particle, or any combination thereof.

18. A method of monitoring hygiene compliance comprising:
applying a hygiene monitoring system as defined in to claim 11 to a surface area of an object; and
detecting the presence or absence of color on the surface area washed with the washing agent.

19. The method according to claim 18, wherein the washing agent and coloring agent are mixed together immediately prior to or during application the surface area.

20. The method according to claim 18, wherein the object is a structure, an article of manufacture or surface from an animal.
